(19) United States
(12) Patent Application Publication
(10) Pub. No.: US 2018/0105787 A1
(43) Pub. Date: Apr. 19, 2018

(54) INCUBATOR SYSTEM

(71) Applicant: Hardin Scientific, Inc., Wildomar, CA (US)

(72) Inventor: Aaron J. Hardin, Wildomar, CA (US)

(21) Appl. No.: 15/786,882

(22) Filed: Oct. 18, 2017

Related U.S. Application Data
(60) Provisional application No. 62/410,212, filed on Oct. 19, 2016.

(51) Int. Cl. C12M 1/00; C12M 3/00; C12M 1/36; B01L 7/00

(52) U.S. Cl. CPC C12M 41/14; C12M 23/44; C12M 41/48; C12M 23/40; B01L 7/52; B01L 2300/18; B01L 2300/0627; B01L 2300/0861; B01L 2300/10; B01L 2300/14; B01L 2300/027

(57) ABSTRACT

An incubator system includes an incubator having a housing defining a chamber, an input port in the housing of the incubator; and a module having an output port configured to releasable mate with the input port of the housing. The module is configured to perform a function. An incubator system is also provided that includes an incubator having a housing defining a chamber, a peripheral device in the chamber configured to manipulate samples, and a master controller configured to control the incubator and the peripheral device. The peripheral device is at least one of powered wirelessly and communicated with wirelessly.

### BACKGROUND

An incubator is an apparatus used to grow and maintain microbiological cultures or cell cultures. As such, the incubator provides a controlled environment or chamber to maintain optimal temperature, humidity, and other conditions such as the carbon dioxide (CO₂), oxygen (O₂), and/or nitrogen (N₂) content of the atmosphere inside.

These chambers come in a variety of shapes and sizes. Consistent features of incubators include a non-porous enclosure that is easily disinfected or sterilized, some kind of insulation to stabilize temperature fluctuations, and methods to alter parameters/variables such as temperature, humidity, and gas concentrations.

Current laboratory incubators usually provide one function, such as humidity or gas concentration control. If one has a laboratory incubator that controls temperature and one wants to also control gas concentration(s), then the only recourse is to buy a second incubator, directed to the additional function(s). This can be expensive, often costing thousands of dollars. Further, each additional incubator takes up a certain amount of area (“footprint”) in the laboratory. In crowded laboratories, having enough space for all the incubators needed can pose problems. Finally, incubators have a finite life, typically 7 to 10 years, and must be replaced, even if only a part of the incubator fails.

Further, current laboratory incubators provide no interconnection of data generated in the incubator or analysis of the generated data. Thus, it is not possible to manipulate and export data.

Another issue with current laboratory incubators involves the use of peripheral equipment, such as shakers, roller apparatus, wave tables and sample monitoring devices. Such incubators may require opening a door to place a peripheral device inside, or may need to leave the door partially open to accommodate a power plug or connector for external connection. However, opening the door or leaving it partially open during the incubation period may expose laboratory personnel to dangerous pathogens that are being grown or maintained in the incubator.

### BRIEF DESCRIPTION OF THE DRAWINGS

The accompanying drawings, which are incorporated in and form a part of the Detailed Description, illustrate various embodiments of the subject matter and, together with the Detailed Description, serve to explain principles of the subject matter discussed below. Unless specifically noted, the drawings referred to in this Brief Description of Drawings should be understood as not being drawn to scale. Herein, like items are labeled with like item numbers.

FIG. 1 depicts an example block diagram of a modular incubator system, according to some embodiments.

FIG. 2 depicts an example block diagram of the elements comprising a modular incubator system, according to some embodiments.

FIG. 3 depicts an example block diagram of a gas module used in the modular incubator system, according to some embodiments.

FIG. 4 depicts details of the electronics of the gas module of FIG. 3, according to some embodiments.

FIG. 5 depicts an example flow chart for initializing a modular incubator system, according to some embodiments.

FIG. 6 depicts an example flow chart for monitoring the operation of a modular incubator system, according to some embodiments.

FIG. 7 depicts an example flow chart for operating a modular incubator system, according to some embodiments.

### DETAILED DESCRIPTION

### Notation and Nomenclature

Embodiments described herein may be discussed in the general context of processor-executable instructions residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, logic, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems described herein may include components other than those shown, including well-known components.

Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As is employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration.

Wired or wireless interconnections may be employed, as appropriate. Networks may employ a network interface, such as a Local Area Network (LAN), a Wide Area Network (WAN), a wireless 802.11 LAN, a 3G or 4G WAN or WiMax WAN and a computer-readable medium. Each of these components may be operatively coupled to a bus, such as EISA, PCI, USB, FireWire, NuBus, or PDS.

It is to be noted that, as used in this specification and the appended claims, the singular forms “a,” “an,” and “the” include plural referents unless the context clearly dictates otherwise. It is appreciated that, in the following description, numerous specific details are set forth to provide a thorough understanding of the examples. However, it is appreciated that the examples may be practiced without limitation to these specific details. In other instances, well-known methods and structures may not be described in detail to avoid unnecessarily obscuring the description of the examples. Also, the examples may be used in combination with each other.

### Overview of Discussion

The discussion that follows is divided into two aspects. The first aspect describes incubators provided with modularity, wherein each component commonly used in incubators is provided as a module to be connected to the incubator. Examples of such modularized components include temperature control, humidity control, and other conditions such as the carbon dioxide (CO₂), oxygen (O₂), and/or nitrogen (N₂) concentration of the atmosphere inside. Further, the Internet of Things (IoT) may be used to communicate data and other information to a remote user and to receive signals and communications from the remote user.

The second aspect of the discussion describes powering and/or communicating with peripherals inside the incubator during its operation. Examples of such peripherals include shakers, roller apparatus, wave tables and sample monitoring devices, among others. In this aspect, the passage of power cords and connector cables through the incubator door is eliminated, thereby protecting laboratory personnel from the possibility of exposure to noxious, dangerous or poisonous contents of the incubator.

### Modularization of Incubator

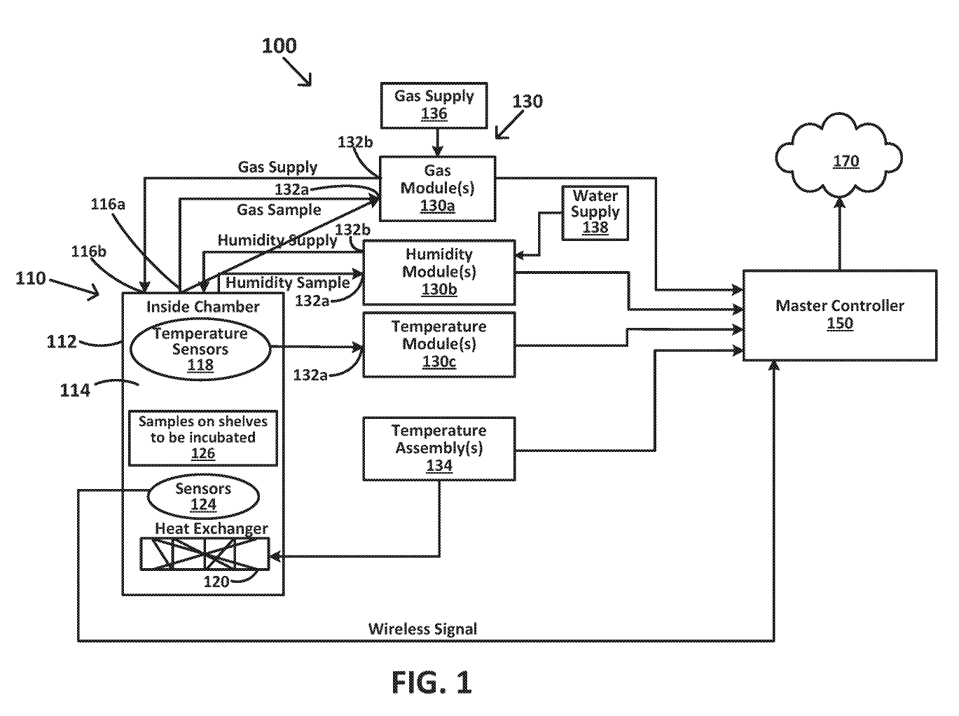

In accordance with aspects of principles disclosed herein, a modularized incubator system is provided. FIG. 1 depicts an example modularized incubator system 100. The incubator system 100 includes an incubator 110 having a housing 112 defining a chamber 114. The incubator system 100 further includes a module 130 having at least an input port 132a. Each module 130 is configured to perform a function, such as gas concentration control of a gas, e.g., CO₂, O₂, N₂ (e.g., module 130a), humidity control (e.g., module 130b), or temperature control (e.g., module 130c). Some modules, such as the gas control module 130a and the humidity control module 130b, also have an output port 132b. The incubator 110 has an output port 116a in the housing 112 that is configured to provide an appropriate sample to the input port 132a of gas module 130a and humidity module 130b and to provide temperature information from a temperature sensor 118 to the temperature module 130c. The incubator 110 also has an input port 116b in the housing 112 that is configured to accept the output from an output port 132b of gas module 130a and humidity module 130b.

The incubator 110 may be configured as any of an oven to provide heat, a refrigerator to provide cooling, and an environmental chamber to provide specific environmental conditions, or a combination of any of these. The incubator 110 may include a ceramic insulating layer in the unit, as described later below.

There may be a plurality of modules 130, each performing a separate function. Examples of the modules 130 and their function include:

a CO₂ module 130a configured to supply CO₂ gas at a desired concentration in the chamber 114;

an O₂ module 130a configured to supply O₂ gas at a desired concentration in the chamber 114;

an N₂ module 130a configured to supply N₂ gas at a desired concentration in the chamber 114;

a humidity module 130b configured to supply water vapor at a desired concentration in the chamber 114; and

a temperature module 130c configured to control temperature in the chamber 114.

The gas module 130a receives gas from a source 136 of the gas and sends the gas to the chamber 114. The humidity module 130b receives water from a water supply 138, converts the water to steam/vapor, and sends the steam/vapor to the chamber 114.

The temperature module 130c communicates with a temperature assembly 134, which may be separate, as shown, or combined into one unit. The temperature assembly 134 controls a heat exchanger 120 in the incubator 110. The temperature assembly 134 is described further below.

The modules 130 are interchangeable with each other; all are provided with the same fittings to mate with corresponding fittings in the incubator 110. Further, each module 130 can be upgraded or replaced without having to also replace the incubator 110. Thus, as modules 130 age or deteriorate, replacement of a module does not require replacement of an incubator 110 that may be still in fine operating condition. Likewise, if the incubator 110 deteriorates, it may be replaced without also having to replace the modules 130.

The incubator system 100 further includes a master controller 150 for controlling the incubator 110 and each module 130 as well as for receiving data from the incubator and the modules. Communication with the incubator 110 and modules 130 may be made over wired or wireless interfaces.

The master controller 150 may communicate with the cloud 170 to provide an Internet of Things (IoT). As is well-known, the IOT is a network of physical devices and other items embedded with electronics, software, sensors, actuators, and network connectivity which enable these objects to collect and exchange data.

The IoT allows objects, here, the components of the incubator system 100, to be sensed or controlled remotely across existing network infrastructure, creating opportunities for more direct integration of the physical world into computer-based systems, and resulting in improved efficiency, accuracy and economic benefit in addition to reduced human intervention. Thus, any data collected by the master controller 150, including settings of sensors and devices and data received from sensors and devices, may be available to a remote user, who can not only view such information, but also make changes in settings in the incubator 110 and modules 130, for example. Essentially, the remote user can perform the same activities remotely as if the remote user were physically present in the laboratory, including manipulating and analyzing data remotely.

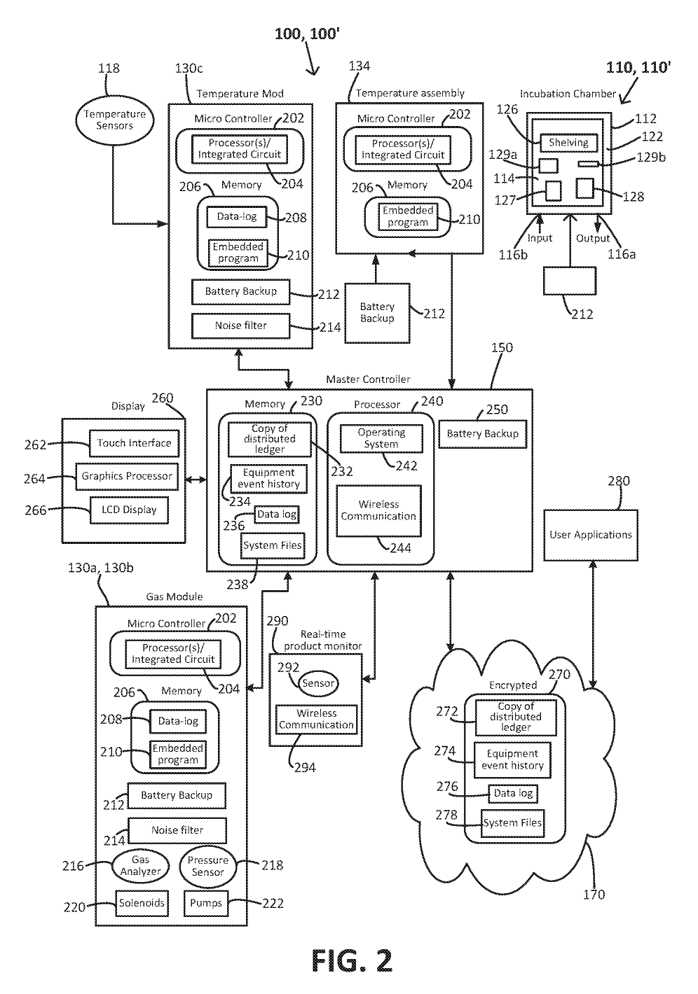

FIG. 2 is a more detailed view of the incubator system 100 depicted in FIG. 1, including the incubator 110, the modules 130a, 130b, 130c, the temperature assembly 134, and the master controller 150. The humidity module 130b has the same elements as gas module 130a, and thus both modules are represented as module 130a, 130b.

In an embodiment, the incubator 110 has the following features, of which some are shown in FIG. 1, some of which are shown in FIG. 2, and some of which are shown in both FIGS. 1 and 2. The incubator 110 comprises chamber 114 defined by stainless steel housing 112 with a ceramic insulator 122, multiple-point temperature mapping via one or more temperature sensors 118 (FIG. 1) that report to the temperature module(s) 130c, various sensors 124 (FIG. 1) that report to the master controller 150, shelving system 126 for supporting samples to be incubated, and the heat exchanger 120 (FIG. 1) that receives input from the temperature assembly(s) 134. In some embodiments, the ceramic insulator 122 that insulates the chamber 114 may a sprayed-on ceramic coating, such as alumina, coated with an aero-gel, such as dehydrated silica.

The incubator 110 further includes an induction coil 127 that wirelessly powers peripheral devices 128 inside the chamber 114, and wireless communications to communicate with other units and devices inside the chamber and the modules 130 to control carbon dioxide, oxygen, nitrogen, relative humidity, temperature and extract various samples from the chamber for quantification/analysis. In an embodiment, peripheral devices 128 inside the chamber 114 may comprise roller apparatus, shakers, wave tables and sample monitoring devices, among others, described in greater detail below. Wired power may be provided by one or more power plugs 129a, while wired communications may be provided by one or more communication receptacles, such as USB ports 129b.

All the components inside the incubator 110 are low power components and are capable of being powered by a small backup battery 212 for several months without external power. The incubator 110 is capable of operating and controlling the gas concentrations for several months without further intervention from the master controller 150 or from any outside source. Therefore, they are said to be capable of operating “headless.” In an embodiment, these modules 130 can also be adapted to fit in existing equipment besides the initial embodiment of laboratory incubators.

The gas module 130a receives gas from a source 136 (FIG. 1) of the gas and sends the gas to the chamber 114. The gas module 130a also extracts a gas sample from the chamber 114 and is in electrical communication with the master controller 150. For example, the gas module 130a communicates the concentration of the gas in the chamber 114 to the master controller 150, while the master controller activates and deactivates the gas module and turns the gas supply 136 on and off through the gas module as appropriate.

The humidity module 130b receives water from a water supply 138 (FIG. 1), converts the water to steam/vapor, sends the steam/vapor to the chamber 114, extracts a humidity sample from the chamber, and is in communication with the master controller 150. For example, the humidity module 130b communicates the humidity level in the chamber 114 to the master controller 150, while master controller activates and deactivates the humidity module and turns the water supply 138 on and off through the humidity module as appropriate.

The temperature module 130c receives information from the temperature sensor(s) 118 that are inside the chamber 114, sends information to the temperature assembly 134 and is in electrical communication with the master controller 150. The temperature assembly 134 controls the heat exchanger 120 (FIG. 1) located inside the chamber 114, receives data from the temperature module 130c, and is in electrical communication with the master controller 150. As noted above, the temperature inside the chamber 114 may be heated or cooled or otherwise conditioned; this is accomplished by the heat exchanger 120 under the control of the temperature assembly 134.

As seen above, the master controller 150 is in electrical communication with all modules 130 and temperature assembly(s) 134, receiving data from each module and assembly and controlling the function of each module and assembly. The master controller 150 is also in communication with a web-based platform via the cloud 170.

Each module 130 and temperature assembly 134 includes a micro controller 202 comprising one or more processors/integrated circuit 204. Each module 130 and temperature assembly 134 further includes a memory unit 206 that includes a data log 208 to store measured data and an embedded program 210 that provides operating instructions for the module 130. Each module 130 and temperature assembly 134 further includes a backup battery 212 in the event of power loss. Each module also includes a noise filter 214 to condition signals to and from the master controller 150.

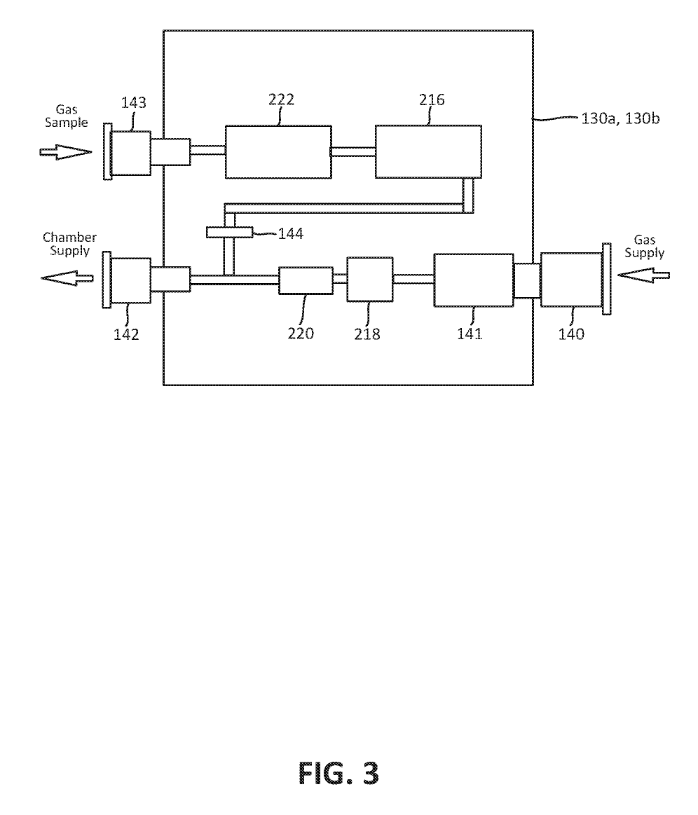

The gas module 130a, 130b further includes a gas analyzer 216 and a pressure sensor 218, along with solenoids 220 and pumps 222. FIG. 3 depicts further details, including the overall function, of the gas module 130a, 130b. The gas from the gas supply 136 (or water vapor from the water supply 138) enters the module 130a, 130b using a quick disconnect fitting 140, goes through an inline regulator 141, is measured by the pressure sensor 218, passes through the solenoid 220 to another hose that has a quick disconnect 142, whereupon this disconnect connects to the chamber 114.

Alternatively, the gas may pass through a heating assembly, picking up conductive heat, and enter a chamber plenum at a reasonable temperature. “Reasonable temperature” may be defined as not having an overall effect on the overall chamber temperature. A heating assembly fan circulates the gas through the chamber plenum and into the chamber 114.

There is another hose coming from the chamber 114 (or the heating assembly) to the gas module 130a, 130b that connects to a pump 222 that mechanically pulls a sample from the inside of the incubator 110 via a quick disconnect 143 and pushes the sample across a gas analyzer, or sensor, 216 to determine the gas concentration along with temperature and humidity. The sample gas is returned to the gas input line after passing through a filter 144.

The gas analyzer 216 calculates the concentration of the gas mixture and data logs that information for up to 2 weeks at 15-minute intervals, for example. This data is processed by the module's micro controller 202 (FIG. 2) where it can be stored for additional lengths of time until there is a serial connection to send the data to the master controller 150.

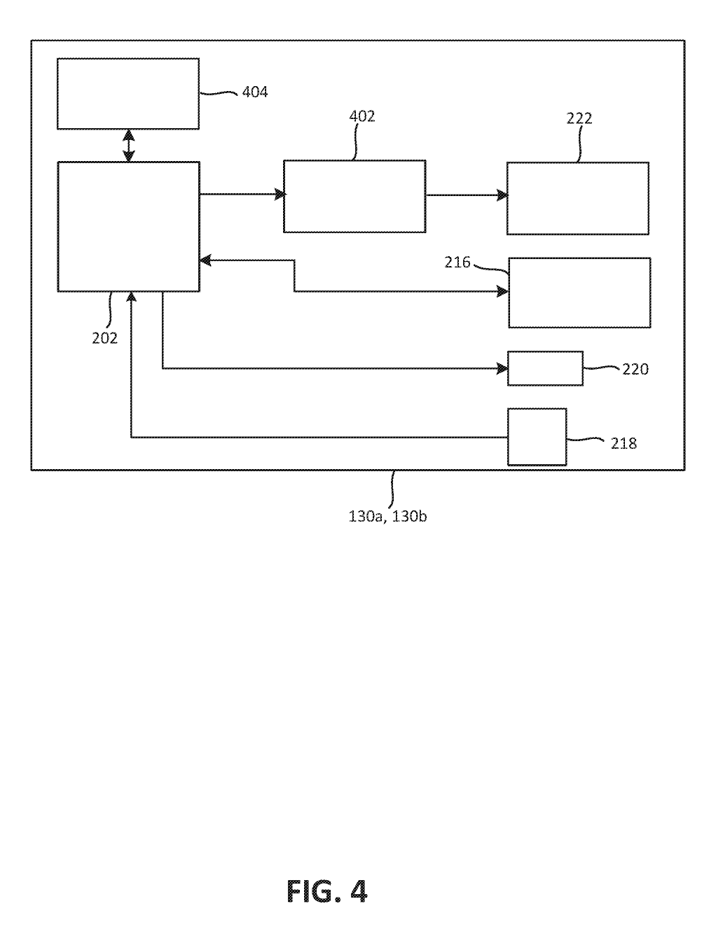

The electronic connections for the gas module 130a, 130b are depicted in FIG. 4. The micro controller 202 is electrically connected directly to the gas analyzer 216, the pressure sensor 218, and the solenoid 220. The micro controller 202 is indirectly connected to the gas sensor 216 through a relay 402. The micro controller 202 is also connected to the master controller 150 by means of a serial connector 404.

Returning to FIG. 2, the overall function of the temperature module 130c is as follows. The temperature is measured by a plurality of temperature sensors 118 which utilize a quick disconnect connector. The connector provides an air-tight fitting to the wall of the chamber 114; the wire goes to a centralized printed circuit board 204 and is converted into a temperature value. This value is then logged and stored on a ledger, or data log, 208, for up to several weeks, awaiting serial connection to be sent to the master controller 150. The master controller 150 takes this log and adds it to a central ledger and displays the real-time temperature and history. This value is also communicated with the temperature assembly 134.

The overall function of the temperature assembly 134 is as follows. The temperature assembly 134, having a set point sent from the master controller 150 from a previously determined user input, compares the value of the real-time temperature from the temperature module 130c to the set point. The temperature assembly 134 controls the amount of voltage and amperage that is applied to the heat exchanger 120 in the chamber 114. An example of the heat exchanger 120 is a Peltier heating/cooling assembly. An algorithm is used that determines a rate of heating or cooling to prevent overshooting the set point in the event of heating or undershooting the set point in the event of cooling.

As the temperature approaches the set point, the temperature assembly 134 maintains a temperature difference great enough to maintain uniform heating or cooling and to counteract the heat loss from the insulated chamber 114. The temperature assembly 134 consists of a micro controller 202 with processor(s)/integrated circuit 204, a heat sink on the outside with plurality of fans to ensure air flow over the heatsink, Peltier heating/cooling units 120 (FIG. 1) to heat or cool which are sandwiched between the external heatsink and a heatsink on the inside of the chamber 114, and a plurality of fans on the internal heatsink to circulate air and pressurize the plenum. The internal fans are resistant to extreme environmental conditions to ensure operation at very high humidity.

There is a high-temperature gasket around the edge of the heatsink to create an airtight barrier between the heat sink in the chamber 114. The heating assembly 134 has a quick disconnect feature to allow the unit to be disconnected in the event maintenance is required, and there is a 120-volt power supply into the heating assembly and quick disconnect fittings for gas lines.

The gas lines connect to a tube that protrudes from the inside heatsink through the external heatsink. The heat sink also has an alternate power source for quick connection of UV lamps or additional heating element(s) for superheating capabilities. The incubator 110 has the capability to include a battery supply 212 to enable the unit to continue operating for several months without an external power source. In the event of a power outage, the incubator is capable of powering the master controller. Other incubator characteristics are as follows.

In some embodiments, the door to the incubator 110 comprises a carbon fiber shell with a touchscreen LCD display, the main controller, a heating element on the interior wall of the door to prevent condensation, hinges that are capable of being quick disconnected, and a bolt lock system to enable the unit to be locked and secured protecting vital samples, equipment, chemicals, hazardous materials, bio-hazardous materials, etc.

The master controller 150 has a positioning sensor such as a MEMS accelerometer or a gyroscope to determine how level the instrument is, can give a graphical display of the unit's levelness, and in the event the unit is on a compatible dolly system, it can adjust the four corners to self-level. The exterior of the master controller 150 consists of a chemically-resistant carbon fiber shell and various LEDs for visual message indication such as unit status, power status, the type of materials within the unit, etc. On all corners, top, sides, bottom, and back are threads to enable the modules 130 to be locked onto one another for stacking or additional stability. Threads also enable hanger rail attachment for the modules 130.

The modules 130 can be attached to the side, top, back, or bottom of the incubator 110. The incubator 110 can be inverted 180 degrees to enable the door to open right to left or left to right or rotated 90 degrees to enable the door to open from bottom to top or from top to bottom. The incubator 110 can also be flipped onto its back to enable the incubator to act as a chest where the door opens upward. In this case, the incubator 110 may require a dolly system to prevent the heating assembly 120 (normally located on the back of the unit) to have proper clearance from the floor for circulation around the heatsinks. In this event, the incubator 110 is oriented as a chest style incubator and stacked on top of another unit; the dolly system is still used but the casters are taken off and secured to the four corners of the incubator below.

The shelving system 126 may act as a shell within the chamber 114 and may be completely self-contained. The shelving system 126 may work as a drawer sliding into the incubator 110 on rails capable of withstanding high temperatures that are secured to the shelving system. The shelves within the shelving system 126 can be repositioned to enable different heights. A sectional glass door may be fixed to the shelving system. There may be handles on either side of shelving system 126 so that the system can be grasped by both sides and pulled out in one piece. The lightweight stainless steel construction enables the shelving system 126 to be removed by a single individual. Other shelving system options are possible and may be interchangeable. In one example, the shelving system 126 can consist of trays that are pulled out to maximize utilization of the entirety of the chamber 114 with optional bisectional glass.

The components that can give real-time data within the chamber 114 can consist of small sensors 124 (FIG. 1) the size of coins with a radio frequency transmitter that sends a signal to the master controller 150. In other situations, the sensors 124 can comprise the entirety of the chamber 114 and consist of extremely complex circuitry, with varying power requirements and radio communications to and from the master controller 150. The sensors 124 can measure the individual samples or the ambient conditions inside the chamber 114.

With continuing reference to FIG. 2, the master controller 150 includes a memory 230, a processor 240, and battery backup 250. The memory 230 includes a copy of a distributed ledger 232 received from each module 130 and temperature assembly 134, an equipment event history 234 received from each module and temperature assembly, a data log 236 received from each module and temperature assembly, and system files 238. The processor 240 includes the operating system 242 and wireless communication components 244.

The master controller 150 graphically displays the event history of the incubator 110 and the real-time data from the modules 130. A display 260 associated with the master controller 150 includes a touch interface 262, a graphics processor 264, and a display 266, such as an LCD display.

The master controller 150 is also responsible for receiving user input and transmitting that data to the modules 130 and temperature assembly 134. In the event of a power outage, an energy saving mode can be initiated and the master controller 150 may power up enough to send the minimum commands to the modules 130 and temperature assembly 134, receive from the modules the data logs 208, and store the logs in the common ledger 232 within the internal memory 230 of the master controller. If power is out for a substantial amount of time, then the master controller 150 can begin eliminating less important functions and lengthen the amount of time between transactions. In the event that the samples inside the chamber 114 are dangerous, a final initiative may reserve enough power to superheat the chamber for a duration of time deemed necessary to destroy any samples within.

The master controller 150 is capable of starting a validation procedure where all of the functions of the incubator 110 are checked. The incubator 110 also performs periodic systems checks and diagnostics known as the “health monitoring system”. All of the histories of the incubator 110 are stored on the common ledger 232 that is encrypted and uploaded via the cloud 170. The upload may be to a distributed decentralized database 270, for example. This record can be accessed by a desktop or mobile application following user authentication. The decentralized data base 270 may include a copy of the distributed ledger 272, a history of equipment events 274, a data log 276, and system files 278.

Reports can be scheduled and generated to report history consisting of the simplest data, to detailed reports about all of the systems operations. A user 280 can also use the desktop or mobile applications to adjust parameters or control the incubator 110 remotely in an IoT environment via the cloud 170. Alarms and notifications customized and sent to the user 280 in the form of an auditory alarm, an email, text, visual indicators, and/or automated phone calls.

The incubator 110 can be calibrated by putting a standard inside the unit that checks gas levels, temperature, relative humidity, and all other variables and communicates any differences with the master controller 150. The “before” measurements may be displayed and the incubator 110 may be adjusted autonomously. The “before and after” measurements may be displayed and stored on the ledger 232. A detailed report may be generated showing the “before”, the “after”, standard deviation, and percent error. Alternatively, the individual modules 130 can be removed and calibrated independently of the incubator 110 in a controlled environment. These results may be uploaded independently to the decentralized database 270.

A real-time product, or sample, monitor 290 may be present to communicate with the master controller 150. The real-time product monitor 290 includes a sensor 292 and wireless communications 294. The purpose of the real-time sample monitoring is to give detailed analytics of sample viability and status during incubation. Parameters of real-time sample monitoring may include pH, particle concentrations such as dissolved oxygen and CO₂, mass spectrometry, cell count, visual monitoring, and temperature.

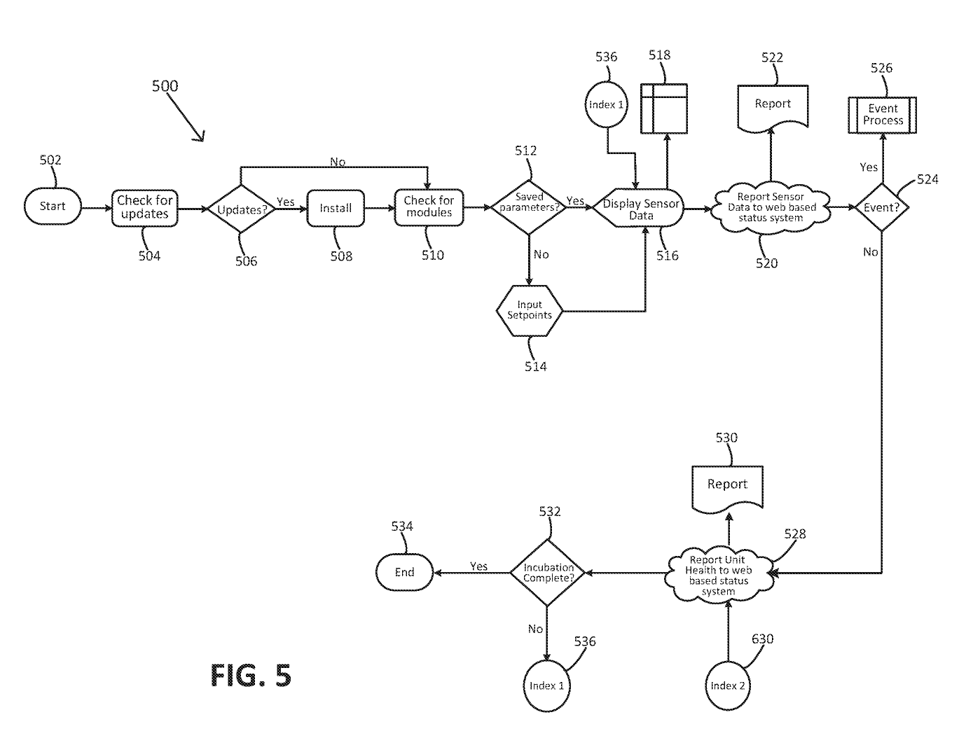

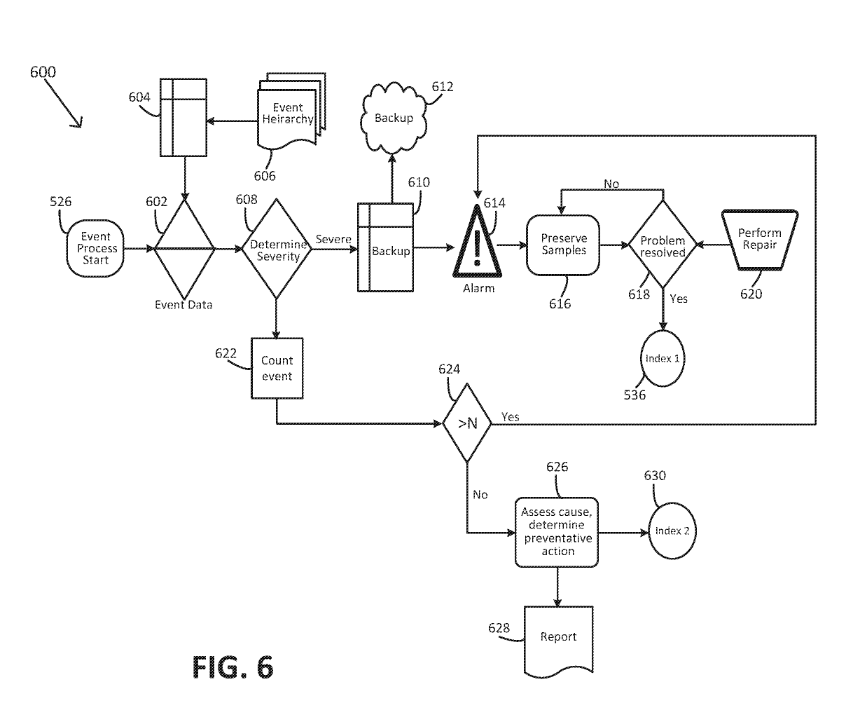

An example of the operation of the master controller 150 is presented in FIG. 5 as a process flowchart 500. Upon starting 502, the master controller 150 checks 504 for updates. If updates are available 506, then a program installs 508 updates and then checks 510 for attached modules. Otherwise, if updates are not available, then the program skips the installation step 508 and begins checking 510 for the modules. The program queries 512 if there are saved parameters for the attached modules. If not, then the program initializes 514 a setup program to input the necessary set points and parameters and then returns to normal operation 516. Otherwise, if parameters are available, then the program begins normal operation 516. In normal operation 516, the program displays 516 the sensor data and stores the data to internal memory 518. This sensor data is uploaded to a web-based status system 520 that generates a report 522. The master controller 150 then checks 524 for “events,” and if there is an “event”, then the unit goes to the event process flow 526 (FIG. 6). Otherwise, the program uploads the unit health to the web-based status system 528 that generates a report 530. If incubation is complete 532 at this point, then program ends 534. Otherwise, the process returns to a previous point in the program indicated by index 1 536.

Turning now to FIG. 6, an example of an event flowchart 600 is depicted. When the event program 600 is initiated 526, the program accesses 602 internal memory documentation 604. This documentation 604 lists protocols and history 606 for various scenarios to determine the severity of the event. If the event is classified 608 as a severe event, all program files are backed up 610 to internal storage and uploaded to a wireless database 612, whereupon the program initiates 614 alarms and preserves 616 the samples. If the problem is resolved 618 by a manual intervention or the problem is self-resolved such as by a repair 620, then the system returns to an operating point on the master control flow diagram 500 determined by the index marker 1 536. Otherwise, the system continues to preserve 616 samples.

Alternatively, if the event is assessed 608 to be a non-severe event, then a counter 622 determines the frequency of occurrence. If the frequency is below a set value N 624, then the program will assess the cause and determine preventative action at 626, generate a report 628, and then return to index 2 630 on the master control flow diagram 500. Otherwise, if the frequency is not below the set value, i.e., >N 624, then the program escalates the report of the severity of the event to alarm 614.

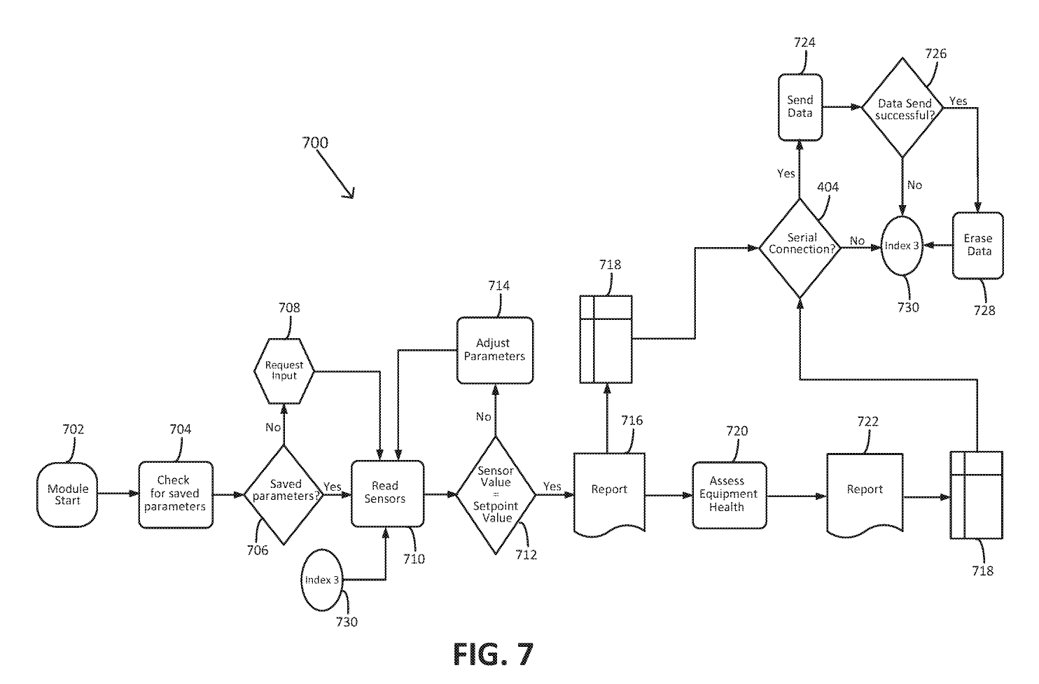

Turning now to FIG. 7, an example of a module controller flowchart 700 is presented. The module 130 starts 702 and checks 704 for saved parameters. If there are no saved parameters 706, then the program initializes a setup program to request input 708 to determine the necessary parameters. Otherwise, the program begins reading 710 sensors. The sensor value is compared 712 to the set point value. If the two are not equal, then the module begins to adjust the parameter 714 and returns to reading the sensor 710. If the sensor value does equal the set point value, then the program reports 716 the previous values adding to the internal memory 718. The program assesses 720 the health of the module and generates a report 722 that gets uploaded to the internal memory 718. If there is a serial connection 404, then the data will be sent 724 to the master controller 150. If data is sent 726 successfully, then the internal memory is erased 728 and the program returns to index 3 730 indicated at an earlier point within this diagram. If the data send 726 is not successful or no serial connection 404 is available, then the program returns to index 3 730 without erasing the data.

### Peripherals

As briefly described with reference to FIG. 2, one or more peripheral devices 128 may be placed in the incubator 110. To avoid having power cords and/or connector cords passing out through the incubator door (a common practice at present), the inductor 127 may be placed in the incubator, along with a wireless communication device, to power the peripheral device 128 and communicate wirelessly with it. Alternatively, powered receptacles 129a and wired communication ports 129b may be provided in the incubator 110 for hard wiring the peripheral device 128.

Examples of peripheral devices 128 that are likely to have at least one of a power cord and a connector cord are listed below. These are all mechanical devices: orbital shaker, vibration shaker, rolling rack, scale, autosampler, robotic arm, and egg/vial turner. Other peripheral devices 128 may be placed in the incubator 110 as appropriate and may be powered and/or communicated with as now described. Thus, the mechanical devices 128 can be seen to manipulate samples, such as by shaking, mixing, rotation, and the like.

Such peripheral devices 128 may need power and may also require instructions to be communicated to them, such as control parameters. These peripheral devices 128 may also generate information that needs to be communicated to the master controller 150. Wireless power and/or communication is one approach, such as using a bluetooth interface. Alternatively, wired power and/or communication may also be employed.

In accordance with aspects of principles disclosed herein, an incubator system 100′ is provided. The incubator system 100′ includes an incubator 110′ having the housing 112 defining the chamber 114. The peripheral device 128 may be placed in the chamber 114, where the peripheral device is configured to manipulate samples. The peripheral device 128 is at least one of powered wirelessly and communicated with wirelessly. The incubator system 100 further includes the master controller 150 configured to control the incubator 110′ and the peripheral device 129.

The modules 130 and other components described above in connection with FIGS. 1 and 2 may be employed in connection with the incubator system 100 and the peripheral device(s) 128.

### Method

In accordance with aspects of principles disclosed herein, a method for building/assembling an incubator 110 for a laboratory comprises providing a sealable housing 112 defining a chamber 114, with an access door and with interior shelving system 126 for receiving micro-organisms to be incubated. The method further comprises providing a main system processor 240 for controlling a plurality of modular subsystems 130 via wirelessly or wired. The main system processor 240 comprises a wireless communications link 244 for communications with select modules. A master controller 150 receives module operating condition status reports and other sensor data, and a master control interface sets operating conditions in the incubator 110. The master controller 150 delivers status reports to a web-based status reporting system 270 via an internet connection through the cloud 170.

The modules 130 further comprise a gas control module 130a to manage gasses in the chamber; a humidity control module 130b to measure and manage chamber humidity; a temperature module 130c to measure temperature of the chamber 114; and temperature assembly 134 to manage chamber temperature. The gas control module 130a includes a gas analyzer 216 to analyze the gas characteristics. A wireless induction power supply system 128 may be included in the chamber 114 for powering select peripheral devices 128.

Each module 130 further reports operating conditions to the master controller 150 and includes a backup battery 212 for fail-safe operation and a plug-compatible standard interface for operating with the master system processor.

### FURTHER CONSIDERATIONS

The modules 130 may conveniently be plug and play boxes that are available in the after-market. Each module 130 has embedded instructions for use. For example, the CO₂ module 130a is pre-programmed to provide CO₂, notifies the master controller 150 when the module is plugged into the incubator 110, and can be programmed for various parameters. The same features are used in the O₂ and N₂ modules 130a and the humidity module 130b.

An opening in back of the chamber 114 houses a heat sink with a neoprene gasket and a fan for circulating air. The heat sink with neoprene gasket can be removed and replaced with another module for controlling a different heat range, using, e.g., thermoelectric elements such as Peltier elements for cooing or nichrome wire elements for heating. Thus, one heating element can be exchanged with another.

A gas module 130a controls gas pressure with a regulator. An integral check valve may be provided to close the gas supply in the event of a leak. Each module 130 is powered with both AC (12 v) in and out and DC (5 v) in and out, plus two serial pins, such as USB for communications. In using a module 130, it is plugged into the incubator 110. Gas supply, e.g., CO₂, is added to the back of module 130a, 130b. The temperature module 130c is plugged into the module 130a, 130b to condition the gas entering the chamber 114.

Every module 130 has a micro controller 202. Thus, every module 130 can be operated independently. The micro controller 202 is configured to log all data collected. Data can then be reported to the master controller 150 by each module 130 when appropriate.

Each module 130, when plugged into the incubator 110, identifies itself to the master controller 150 as to what it does, along with other information, such as the manufacture date, the calibration date, and instructions to the master controller how to tell the module what to do. The user enters desired changes at the master controller 150 (e.g., a desired CO₂ level), and the master controller sends a message to the module to change its set point. A Graphic User Interface may be employed. The user can access all calibration histories, maintenance histories, and data points for a single module 130 or for all modules and bring that data up on a mobile or desktop app.

To retrieve data from the master controller 150, the data can be accessed on the unit itself, such as via the display 260 or exported to a printer or mobile device or desktop. The master controller 150 has at least one of a wireless interface, a bluetooth interface, or a wired ethernet connector. There is ordinarily one master controller 150 per incubator 110, controlling as many modules 130 as desired. Any changes in parameters of a module 130 may be made through the master controller 150.

An advantage of the incubator system 100 is that real time sample monitoring may be used, due to bluetooth, This means that each sample can be provided with a RFID device such as sensor 124 that communicates with the master controller 150. Consequently, for 100,000 samples in the chamber 114, all samples can be monitored in real time.

Another advantage of the incubator system 100 is that predictive failure analysis of the modules 130 is available. Using predictive failure analysis, it is possible to determine when a module 130 is getting ready to fail well before failure, based on the analytics received by the master controller 150

Yet another advantage of the incubator system 100 is that when an individual module 130 fails, only it, and not the entire incubator 110, can be replaced. This reduces the cost considerably over the long term. Further, a laboratory need only buy one incubator 110 and a plurality of modules 130, using a particular module at any given time. This saves space in the laboratory, since multiple incubators 110 are not necessary.

While only a few embodiments of the present invention have been shown and described, it will be obvious to those skilled in the art that many changes and modifications may be made thereunto without departing from the spirit and scope of the present invention as described in the following claims. All patent applications and patents, both foreign and domestic, and all other publications referenced herein are incorporated herein in their entireties to the full extent permitted by law.

The embodiments and examples set forth herein were presented in order to best explain various selected embodiments of the present invention and its particular application and to thereby enable those skilled in the art to make and use embodiments of the invention. However, those skilled in the art will recognize that the foregoing description and examples have been presented for the purposes of illustration and example only. The description as set forth is not intended to be exhaustive or to limit the embodiments of the invention to the precise form disclosed.

The methods and systems described herein may be deployed in part or in whole through a machine that executes computer software, program codes, and/or instructions on a processor. The present teachings may be implemented as a method on the machine, as a system or apparatus as part of or in relation to the machine, or as a computer program product embodied on the computer readable medium executing on one or more of the machines. The processor may be part of a server, client, network infrastructure, mobile computing platform, stationary computing platform, or other computing platforms. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the processor may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer to peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or intangible items from one state to another. The methods and systems described herein may also transform data representing physical and/or intangible items from one state to another.

The elements described and depicted herein, including in flowcharts and block diagrams throughout the figures, may imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flowchart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable devices, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

What is claimed is:

1. An incubator system including:
an incubator having a housing defining a chamber;
an input port in the housing of the incubator; and
a module having a connection port configured to releasable mate with a connection port of the housing, the module configured to perform a function.

2. The incubator system of claim 1, wherein the module is one of a gas module, a humidity module, and a temperature module and wherein each module has the same connection ports so that all modules are interchangeable.

3. The incubator system of claim 2, wherein the module is one of the following:
a CO₂ module configured to supply CO₂ gas at a desired concentration in the chamber;
an O₂ module configured to supply O₂ gas at a desired concentration in the chamber;
an N₂ module configured to supply N₂ gas at a desired concentration in the chamber;
a humidity module configured to supply water vapor at a desired concentration in the chamber; and
a temperature module configured to control temperature in the chamber.

4. The incubator system of claim 1, wherein the incubator system further includes a master controller to control the module.

5. The incubator system of claim 4, wherein the module has embedded instructions for its use, configured to communicate the use to the master controller.

6. The incubator system of claim 4, wherein the module includes a micro controller configured to log data collected, to store the data, and to communicate the data to the master controller.

7. The incubator system of claim 6, wherein each module further includes a memory and battery backup.

8. The incubator system of claim 1, wherein the incubator is insulated with a ceramic insulator, comprising a ceramic layer coated with an aero-gel.

9. The incubator system of claim 1, further including a peripheral device in the chamber, configured to manipulate samples, the peripheral device being at least one of powered wirelessly and communicated with wirelessly.

10. The incubator system of claim 8, wherein the peripheral device is one of an orbital shaker, a vibration shaker, a rolling rack, a scale, an autosampler, a robotic arm, and an egg/vial turner.

11. An incubator system including:
an incubator having a housing defining a chamber;
a peripheral device in the chamber configured to manipulate samples; and
a master controller configured to control the incubator and the peripheral device, wherein the peripheral device is at least one of powered wirelessly and communicated with wirelessly.

12. The incubator system of claim 11, wherein the peripheral device is one of an orbital shaker, a vibration shaker, a rolling rack, a scale, an autosampler, a robotic arm, and an egg/vial turner.

13. The incubator system of claim 11 further including
a connection port in the housing of the incubator; and
a module having a connection port configured to releasable mate with the connection port of the housing, the module configured to perform a function.

14. The incubator system of claim 13, wherein the module is one of a gas module, a humidity module, and a temperature module and wherein each module has the same connection ports so that all modules are interchangeable.

15. The incubator system of claim 14, wherein the module is one of the following:
a CO₂ module configured to supply CO₂ gas at a desired concentration in the chamber;
an O₂ module configured to supply O₂ gas at a desired concentration in the chamber;
an N₂ module configured to supply N₂ gas at a desired concentration in the chamber;
a humidity module configured to supply water vapor at a desired concentration in the chamber; and
a temperature module configured to control temperature in the chamber.

16. The incubator system of claim 11, wherein the master controller also controls the module.

17. The incubator system of claim 16, wherein the module has embedded instructions for its use, configured to communicate the use to the master controller.

18. The incubator system of claim 16, wherein the module includes a micro controller configured to log data collected, to store the data, and to communicate the data to the master controller.

19. The incubator system of claim 18, wherein each module further includes a memory and battery backup.

20. The incubator system of claim 11, wherein the incubator is insulated with a ceramic insulator, comprising a ceramic layer coated with an aero-gel.
